(19) United States
(12) Patent Application Publication
(10) Pub. No.: US 2009/0043160 A1
(43) Pub. Date: Feb. 12, 2009

(54) SELF-PROPELLED COLONOSCOPE

(76) Inventor: Masazumi Takada, Tokyo (JP)

Correspondence Address: YOUNG & THOMPSON, 209 Madison Street, Suite 500, ALEXANDRIA, VA, 22314, US

(21) Appl. No.: 12/188,256

(22) Filed: Aug. 8, 2008

(30) Foreign Application Priority Data
Aug. 10, 2007 (JP) 2007-209512
Aug. 23, 2007 (JP) 2007-217158
Sep. 25, 2007 (JP) 2007-247274

(51) Int. Cl. A61B 1/31

(52) U.S. Cl. 600/114

(57) ABSTRACT

A self-propelled colonoscope comprises a tubular flexible insertion portion (15) inserted into the colon and an endless belt (17) arranged on a circulating path on a part of the outer wall and the inner wall of the flexible insertion portion. The endless belt (17), supported by the guide hooks (39) mounted on the outer wall of the flexible insertion portion at the distal end portion of the flexible insertion portion (15), travels outside the flexible insertion portion (15). And, the endless belt (17) passes through the guide pipe (43) mounted on the inner wall of the flexible insertion portion (15) at a portion other than the distal end portion. The circulating path on the outer wall of the flexible insertion portion (15) is 10 to 60 cm in length.

### FIELD OF THE INVENTION

The present invention relates to a self-propelled colonoscope to be inserted into the colon by endless driving belts, arranged on the inner and outer walls of a flexible section of an insertion tube in a loop fashion.

### BACKGROUND OF THE INVENTION

Colonoscopic examination is currently carried out by inserting a colonoscope into the colon by hand. Since the colonoscope has to be inserted around the curves of the colon, the examination may cause the patient pain by excessively extending or bending the colon. Sometimes, the colonoscope perforates the colon wall. As a colonoscope which does not cause a patient pain, colonoscopes self-propelled around the curves of the colon have been proposed.

The inventor proposed a self-propelled colonoscope equipped with endless belts arranged on the inner and outer walls of a flexible section of the insertion tube in a loop fashion and to be inserted into the colon by driving the endless belts (Japanese Patent No. 3514252). Outside the flexible section, the endless belts are supported by guide hooks and travel in the opposite direction to the insertion direction of the colonoscope with contacting the inner wall of the colon, propelling the colonoscope into the colon. Inside the flexible section, the endless belts travel through guide pipes, mounted on the inner wall of the flexible section extending in the length direction of the flexible section. By driving the endless belts with a driving mechanism, the endless belts propel the colonoscope into the colon by friction between the endless belts and the inner wall of the colon outside the flexible section. Inside the flexible section, the endless belts travel through the guide pipes smoothly. So the colonoscope can be propelled into the colon without excessive extension and bending of the colon. Therefore, the colonoscope can be smoothly inserted into the colon while keeping the colon in relatively the same position and shape, causing the patient hardly any pain.

As mentioned above, the endless belts are supported by the guide hooks mounted on the outer wall of the flexible section outside the flexible section. In order to support the endless belts without detaching from the flexible section, it is preferred to arrange the guide hooks as short intervals (for example, 1 to several cm) as possible. However, the shorter the interval becomes, the smaller the flexibility of the flexible section becomes.

### SUMMARY OF THE INVENTION

The object of the invention is therefore to provide a self-propelled colonoscope designed to make a portion provided with the guide hooks as short as possible so as not to deteriorate the flexibility of the flexible section.

A self-propelled colonoscope according to the present invention comprises: a tubular flexible insertion portion to be inserted into a colon; an endless belt arranged around a circulating path on the inner wall and a part of the outer wall of the flexible insertion portion; a driving mechanism to drive the endless belt; guide hooks arranged around the circulating path on the outer wall of the flexible insertion portion; and a guide pipe extending along the circulating path on the inner wall of the flexible insertion portion and guiding the endless belt, wherein the circulating path on the outer wall of the flexible section at the distal end portion of the flexible insertion portion is 10 to 60 cm in length.

In the present invention, the circulating path on the outer wall of the flexible insertion portion is set to 10 to 60 cm in length. The reason for setting a maximum length of the circulating path on the outer wall of the flexible insertion portion to 60 cm is as follows. If an effective travel length of the endless belt may be set to about 60 cm which is a sum of a length of free segment i.e. transverse colon and an allowance of 10 cm, sufficient self-propelling ability may be given. “The distal end portion of the flexible insertion portion” means a peripheral region of the distal end and therefore the colonoscope may include a portion 0 to 10 cm in length from the distal end of the flexible insertion portion provided with no endless belt. According to the present invention, since a length of a portion of the flexible insertion portion provided with the endless belts arranged on the outer wall of the flexible insertion portion is set as short as possible, the insertion portion can be inserted while keeping its flexibility.

In the present invention, the endless belt may travel through the guide pipes, arranged on the inner wall of the flexible insertion portion, other than the circulating path on the outer wall of the flexible insertion portion at the distal end portion of the flexible insertion portion.

A self-propelled colonoscope according to the present invention, expressed in another way, comprises: a tubular flexible insertion portion to be inserted into a colon; an endless belt arranged around a circulating path on the inner wall and a part of the outer wall of the flexible insertion portion; a driving mechanism to drive the endless belt; and a guide pipe extending along the circulating path on the inner wall of the flexible insertion portion and guiding the endless belt, wherein the circulating path on the outer wall of the flexible insertion portion extends only along the distal end portion of the flexible insertion portion, and the endless belt travels through the guide pipes forward and backward, arranged on the inner wall of the flexible insertion portion, other than the circulating path on the outer wall of the flexible insertion portion at the distal end portion of the flexible insertion portion.

The guide hooks may be omitted in case that a length of the circulating path on the outer wall of the flexible insertion portion is short.

### EFFECT OF THE INVENTION

As described above, according to the present invention, a length of a portion of the colonoscope provided with the guide hooks is set to 10 to 60 cm from the distal end of the flexible section to the proximal end and therefore a portion in which the endless belts travel on the outer wall of the flexible section is as short as possible. Accordingly, the flexible section can be inserted while keeping the flexibility.

### BRIEF DESCRIPTION OF THE DRAWINGS

FIG. 1 is a perspective drawing showing the self-propelled colonoscope (example) according to one design of the present invention;

FIG. 2 is a sectional drawing showing the distal section of the insertion portion of the colonoscope of FIG. 1;

FIG. 3(A) is a side sectional drawing showing the insertion portion and a part of the driving unit of the colonoscope of FIG. 1; FIG. 3(B) is an enlarged side sectional drawing showing a part of FIG. 3(A); and FIG. 3(C) is an enlarged side sectional drawing showing another part of FIG. 3(A);

FIG. 4(A) is an A-A cross sectional drawing of FIG. 3(A) and FIG. 4(A) is a B-B cross sectional drawing of FIG. 3(A);

FIG. 5(A) is a perspective drawing schematically showing the structure of the endless belt; FIG. 5(B) is a side drawing schematically showing the pulley on which the endless belt is wound; and FIG. 5(C) is a side drawing schematically showing the engagement state of the endless belt with the pulley; and

FIG. 6 is a drawing schematically showing the exemplary colonoscope insertion path.

### DESCRIPTION OF THE PREFERRED EMBODIMENT

Referring to the attached drawings, the details of the design of the present invention will be presented.

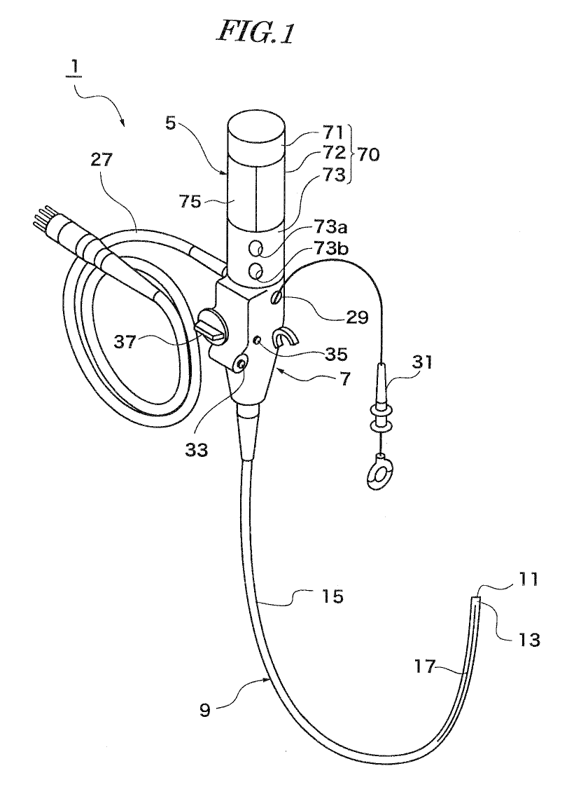

FIG. 1 is a perspective drawing showing the self-propelled colonoscope (example) according to one design of the present invention.

A self-propelled colonoscope 1 is provided with a belt driving unit 5 covered with a driving unit casing 70, an operation unit 7 under the belt driving unit 5, an insertion portion (insertion tube) 9 extending from the operation unit 7 and inserted into the colon, etc. The insertion tube 9 comprises a distal section 11, a bending section 13 and a flexible section (flexible insertion portion) 15. Multiple (three, in this example) endless belts 17 are longitudinally arranged on the flexible section 15.

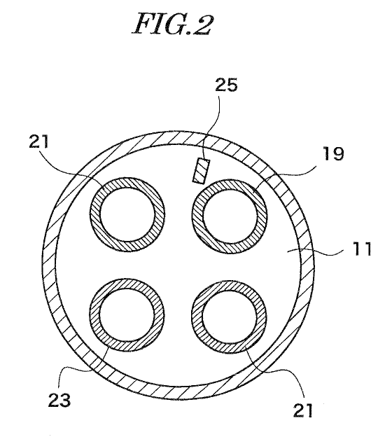

FIG. 2 is a sectional drawing showing the distal section of the insertion portion of the colonoscope of FIG. 1.

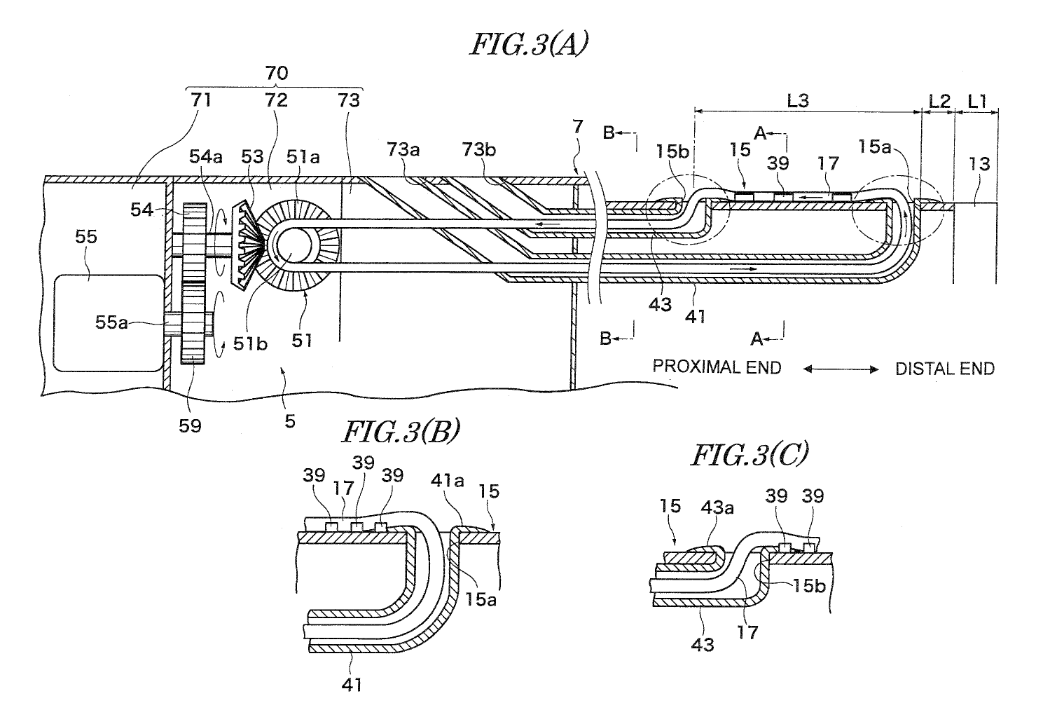

FIG. 3(A) is a side sectional drawing showing the insertion portion and a part of the driving unit of the colonoscope of FIG. 1; FIG. 3(B) is an enlarged side sectional drawing showing a part of FIG. 3(A); and FIG. 3(C) is an enlarged side sectional drawing showing another part of FIG. 3(A).

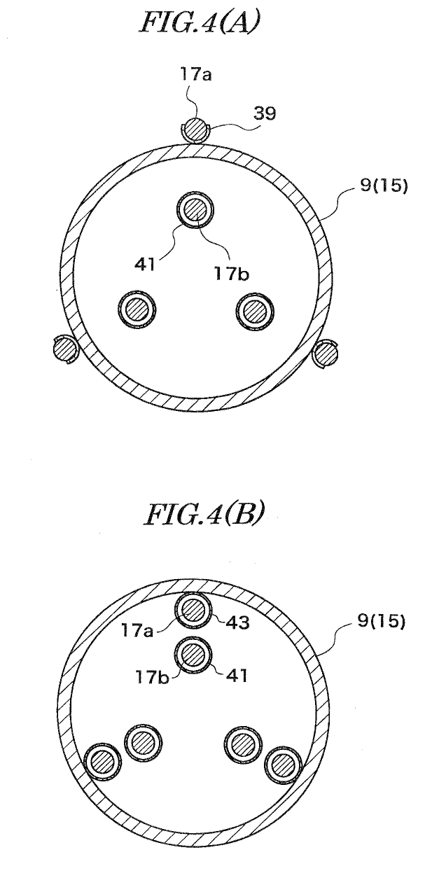

FIG. 4(A) is an A-A cross sectional drawing of FIG. 3(A) and FIG. 4(A) is a B-B cross sectional drawing of FIG. 3(A).

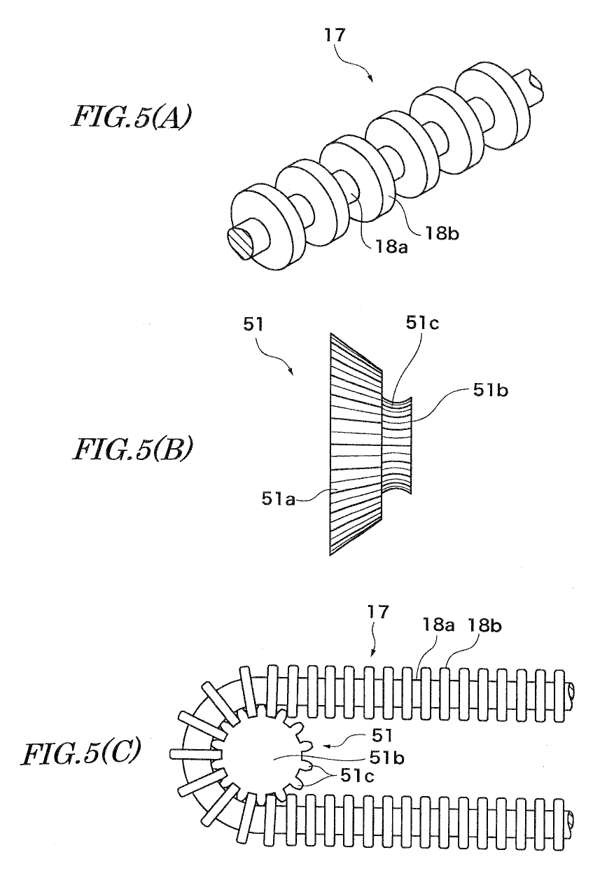

FIG. 5(A) is a perspective drawing schematically showing the structure of the endless belt; FIG. 5(B) is a side drawing schematically showing the pulley on which the endless belt is wound; and FIG. 5(C) is a side drawing schematically showing the engagement state of the endless belt with the pulley.

At the distal section 11 of the insertion tube 9, as shown in FIG. 2, an image receiving window 19, one or two light-projecting windows 21 (two windows in this design), a suction and forceps opening 23, and an air-water nozzle 25 are shown. The image-receiving window 19, equipped with an objective lens when the observation device is a fiberscope, or an image pick-up device, such as a CCD, when it is an electronic scope, receives an image from the distal end surface. The received image is transmitted to the operation unit 7 through the image guide of the fiberscope or the lead wire of the electronic scope, which is inserted into the insertion tube 9, and then transmitted through a universal cord 27 to a display. A light guide, such as an optical fiber, is inserted into the bore of each light-projecting window 21, runs through the operation unit 7 and is connected to an external light source via the universal cord 27. The light source projects light from the distal end surface of the light guide.

The suction and forceps opening 23 is connected to the forceps insertion opening 29 (referring to FIG. 1) in the operation unit 7, and forceps 31 is inserted therein. The working tip end of the forceps 31, protruding from the distal end of the insertion tube 9, is manipulated at the proximal part of the forceps 31 to perform procedures such as to treat a lesion or collecting tissue from a patient.

The bore of the air-water nozzle 25 is a water-air supply channel through which air or a cleaning solution flows and is injected from the air-water nozzle 25 by manipulating the air-water supply button 33 mounted on the operation unit 7. Through the suction and forceps opening 23, bodily fluid or cleaning solution remaining in the colon is sucked out and discharged to outside the patient's body. This operation is carried out by manipulating the suction control button 35 on the operation unit 7.

The flexible section 13 of the insertion tube 9 is able to bend upward and downward, rightward and leftward, and obliquely by manipulating a control knob 37 on the operation unit 7. The flexible section 13 has a length L1 (shown in FIG. 3) of about 10 cm, for example.

As shown in FIGS. 3 and 4, multiple endless belts 17 are longitudinally arranged on the flexible section 15 of the insertion tube 9. The diameter of the flexible section 15 is preferably 5 to 30 mm; within 20 mm is most preferable. In this example, three of the endless belts 17 are arranged. A larger number of endless belts 17 are preferable, because the colonoscope has greater self-propelling property as the number of endless belts increases.

As shown in FIG. 3, the endless belts 17 circulate on a circulating path on the inner wall and a part of the outer wall of the flexible section 15. The circulating path extends between the distal end of the flexible section 15 and a driving roller 51 (described later in detail) of the belt driving unit 5 stored in the driving unit casing 70 in the length direction of the flexible section 15. As described in detail, a forward path of the circulating path on which the endless belts 17 travel toward the distal end of the flexible section 15 from the driving roller 51 is arranged on the inner wall of the flexible section 15. And, a part of a backward path of the circulating path on which the endless belts 17 travel toward the driving roller 51 from the distal end of the flexible section 15 is arranged on the outer wall of the flexible section 15 and the other part is arranged on the inner wall. Since sigmoid colon is about 42 cm in length and transverse colon is about 45 cm in length, the backward path arranged on the outer wall of the flexible section 15 has a length of 10 to 60 cm, more preferably 20 to 60 cm.

In the forward path toward the distal end of the flexible section 15 from the driving roller 51, the endless belt 17 passes through an inner guide pipe 41 arranged on the inner wall of the flexible section 15. The inner guide pipe 41 extends between a guide pipe hole 73a formed at the driving unit casing 70 and a guide hole 15a penetrating through the wall of the distal end of the flexible section 15. As shown in FIG. 3(B), the inner guide pipe 41 has a flange portion 41a at the distal end thereof. The guide pipe 41 passes through the guide hole 15a and the flange portion 41a is fixed on the outer wall. A distance L2 between the distal end of the flexible section 15 and the guide holes 15a is preferably 0 to 10 cm, as shown in FIG. 3(A).

The endless belt 17 enters the inner guide pipe 41 from the driving roller 51 through the wall of the inner guide pipe 41 and travels in the insertion direction of the colonoscope.

In the backward path toward the driving roller 51 from the distal end of the flexible section 15, the endless belts travel on the outer wall of the flexible section 15 initially and then on the inner wall of the flexible section 15. On the outer wall of the flexible section 15, the endless belt 17 is supported by guide hooks 39 mounted on the outer wall of the flexible section 15. On the inner wall of the flexible section 15, the endless belt 17 passes through an outer guide pipe 41 mounted on the inner wall of the flexible section 15.

As shown in FIG. 3, the circulating path on the outer wall of the flexible section 15 has a length L3 of 10 to 60 cm (described later in detail).

The guide hooks 39, each having a circular-arc cross section with a center angle over 180°, are mounted lengthwise along the flexible section 15 at suitable intervals, and support the endless belts 17 to be exposed radially and outwardly from the guide hooks 39, as shown in FIG. 4(A). Accordingly, the outward surface of the endless belt 17 supported by the guide hooks 39 is exposed from the guide hooks 39 so as to be in contact with the inner wall of the colon with a sufficient contact area when the flexible section 15 is inserted into the colon. Even when the flexible section 15 is severely bent, the endless belts 17 will not come off the guide hooks 39.

The guide hooks 39 are mounted lengthwise along the flexible section 15 at intervals of 2 to 3 cm, in this design; however, the guide hooks 39 may be continuously placed in the lengthways direction of the flexible section.

The outer guide pipe 43 extends between a guide pipe hole 73b formed at the driving unit casing 70 and a guide hole 15b formed at a proximal side of the most proximal guide hook 39. As shown in FIG. 3(C), the outer guide pipe 43 has a flange portion 43a at the distal end thereof. The guide pipe 43 passes through the guide hole 15b and the flange portion 43a is fixed on the outer wall.

As shown in FIG. 4(B), the outer and inner guide pipes 43 and 41 extend parallel in the flexible section 15; however, the guide pipes 43 and 41 may be arranged diagonally as shown in FIG. 4(B) or circumferentially.

The endless belt 17 gets out of the inner guide pipes 41 through the guide holes 15a at the distal end portion of the flexible section 15 and turns toward the proximal end, supported by the guide hooks 39 outside the flexible section 15. And then, the endless belt 17 enters the outer guide pipe 43 through the guide hole 15b and travels toward the driving roller 51 in the opposite direction to the insertion direction.

As shown in FIG. 3, when the colonoscope is inserted, the endless belts 17, supported by the guide hooks 39 mounted on the outer wall of the flexible section 15, travel in the opposite direction to the insertion direction while remaining in contact with the inner wall of the colon outside the flexible section 15. This propels the colonoscope into the colon. When the colonoscope is removed from the patient's body, the endless belts 17 circulate in the opposite direction to the direction at insertion. That is, the endless belts 17, supported by the guide hooks 39 outside the flexible section 15 and the outer guide pipes 43 inside the flexible section 15, travel in the insertion direction from the driving roller 51 toward the distal end of the flexible section 15. And, the endless belts 17, supported by the inner guide pipes 41 inside the flexible section 15, travel in the opposite direction to the insertion direction from the distal end of the flexible section 15 toward the driving roller 51.

Each of the endless belts 17 is made of a flexible and strong material such as, for instance, carbon fiber or resin and, as shown in FIG. 5(A) and FIG. 5(C), each comprises an axial belt 18a and multiple rack gear teeth 18b arranged lengthways along the axial belt 18a, which has a circular cross section with a diameter of 1 to 3 mm. The rack gear teeth 18b also have a circular cross section and are coaxially arranged at regular intervals on the outer surface of the axial belt 18a. The rack gear teeth 18b have a diameter of 1 to 3 mm and a thickness of 0.1 to 1.0 mm and the distance between any two of the rack gear teeth 18b is 0.1 to 1.0 mm. The diameters of the axial belt 18a and the rack gear teeth 18b are selected within these ranges so that the diameter of the rack gear teeth 18b is larger than the diameter of the axial belt 18a. The outer surface of the rack gear teeth 18b may be coated with high frictional material, and the outer surface of the pulley 51b including a pinion gear teeth 51c, as described later, may be also coated with high frictional material. The length of the endless belt 17 will be described later.

The endless belt has a circular cross section so as to be able to bend flexibly in all radial directions to the axis with equal force. So, when the insertion tube 9 is inserted around the bends of the colon, the endless belt 17 can easily follow the motion of the insertion tube 9. Since the rack gear teeth 18b are formed along the entire length of the endless belt 17, if the endless belt 17 is twisted, the rack gear teeth 18b may be in contact with the inner wall of the colon, causing the endless belt 17 to rub against it. Accordingly, the friction force between the endless belts 17 and the inner wall of the colon will be increased to improve the self-propelling ability.

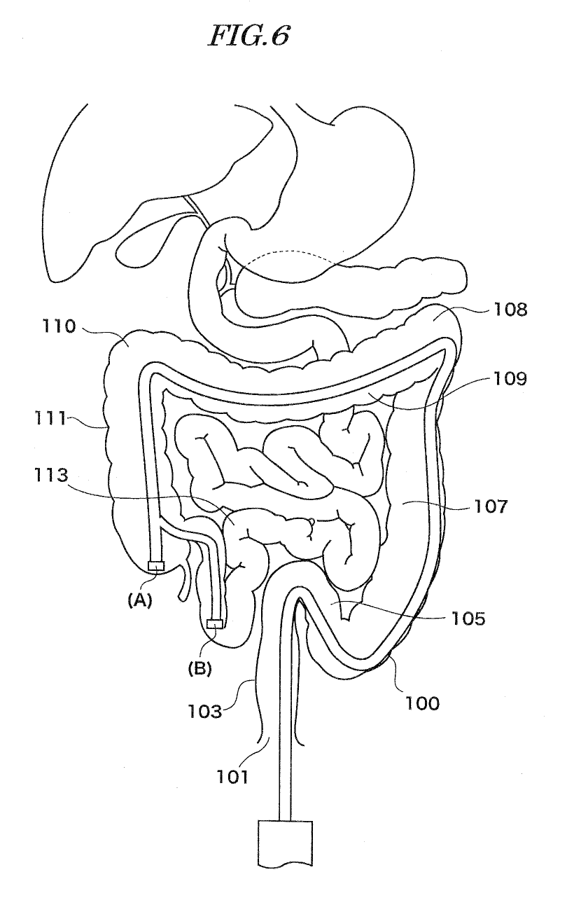

The insertion tube 9, inserted into the colon, advances from the sigmoid colon 105 to the ileum 107 through the descending colon, the transverse colon 109 and the ascending colon 111 as described later referring to FIG. 6. Since the flexible section 15 has an outer diameter as great as about 16 mm, when the colonoscope advances into the colon, the curve of the colon causes a difference in the inner ring length and the outer ring length of the inserted flexible section 15. For example, when the distal end of the insertion tube 9 reaches the ileum and the flexible section 15, having a diameter of 16 mm, turns in a curve, the outer ring length is 3.12% longer than the straight length.

Accordingly, the endless belt 17 arranged on the outer and inner walls of the flexible section 15 necessarily has an allowable length for differences in the inner and outer ring length of the flexible section 15. For this reason, the length of the endless belt 17 is designed be 102 to 104% of the length of the belt when it turns under tension from the guide hole 49 formed at the distal end of the flexible section 15 to the same guide hole 49 through the driving unit while keeping the flexible section 15 straight. Since the length of the endless belt 17 is set as above, it can sufficiently follow the bending of the flexible section 15; therefore, the colonoscope can be propelled into the colon stably.

As mentioned above, the length of the endless belt 17 has a certain allowance; however, since the pulley 51b for driving the endless belt 17 is formed with the pinion gear teeth 51c on the circumferential surface, the endless belt 17 and the pulley 51b are tightly engaged with each other by engagement of the rack gear teeth 18b and the pinion gear teeth 51c, whereby the endless belt 17 can be driven without idling.

As described above, the guide hooks 39 are mounted only along a section between a distal position 0 to 10 cm apart from the distal end of the flexible section 15 and a proximal position 60 cm apart from the distal end of the flexible section 15. The reason will be described below.

An exemplary colonoscope insertion path will be described.

FIG. 6 is a drawing schematically showing the exemplary colonoscope insertion path.

The insertion tube of the colonoscope is inserted from the anus 101 into the rectum 103 (fixed segment) and advances from the sigmoid colon 105 (free segment) to the ileum 113 through the descending colon 107 (fixed segment), the transverse colon 109 (free segment) and the ascending colon 111 (fixed segment). The distal end of the insertion tube may be reached to the terminal of the colon, as shown in (A) in the figure, or to ileum 113 for about 25 cm, as shown in (B) in the figure.

In the case of the present self-propelled colonoscope, when the distal end of the insertion tube 9 enters the sigmoid colon 105, the sigmoid colon 105 moves backward (in the direction of the anus) accompanied with the insertion of the colonoscope so as not to be extended excessively. And, owing to the insertion of the colonoscope and the motion of the sigmoid colon 105, a distance between the distal end of the descending colon 107 and the distal end of the colonoscope becomes short and therefore the sigmoid colon 105 and the descending colon 107 align substantially straight. And, when the distal end of the insertion tube 9 enters the transverse colon 109, the transverse colon 109 moves toward the splenic flexure 108 and the distal end of the insertion tube comes close to the hepatic flexure 110. As the result, the angle of the hepatic angle 110 becomes larger so that the colonoscope can easily pass through the hepatic flexure 110.

On the above mentioned insertion process, a portion of the flexible section 15 provided with the endless belts 17 arranged on the outer wall of the flexible section 15 (that is, a portion of the flexible section 15 provided with the guide hooks 39, in other ward, a portion which provides a propelling force of the colonoscope) may be only a portion of the colonoscope which passes through the free segments (the sigmoid colon 105 and the transverse colon 109). The sigmoid colon 105 is about 45 cm in length and the transverse colon 109 is about 50 cm in length. When the distal end of the colonoscope is inserted into the descending colon 107 for 10 cm, the distal end of the colonoscope should be inserted into the descending colon 107 from the sigmoid colon 105 completely. When the distal end of the colonoscope is inserted into the ascending colon 111 for 10 cm, the distal end of the colonoscope should be inserted into the ascending colon 111 from the transverse colon 109 via the hepatic flexure 110 completely.

As described above, a portion of the flexible section provided with the guide hooks 39 has a length of 10 to 60 cm. The reason for setting the length of the portion to under 60 cm is that if the endless belt 17 has an effective driving length of about 60 cm which is a sum of a length of the transverse colon of a free segment and an allowance of 10 cm, it becomes possible to give sufficient self-propelling force to the colonoscope.

Then, the belt driving unit 5 and the casing 70 in which the belt driving unit 5 is stored will be described referring to FIG. 3.

The belt driving unit 5 is provided with three of gear assembly 50 (one of them is shown in FIG. 3), on which each of the three endless belts 17 is wound, and a motor 55 which drives the gear assemblies 50. The gear assemblies 50 are arranged at a regular angle (60°, in this example) around an output axis 55a of the motor 55.

Each of the gear assemblies 50 is provided with the driving roller 51 around which the endless belt 17 is wound. The driving roller 51 has a pulley 51b on which the endless belt 17 is wound and a bevel gear 43a connected to the same shaft as the pulley 51b. On the circumferential surface of the pulley 51b, as shown in FIG. 5(B), a concave groove is formed, in which the pinion gear teeth 51c are fixed, which engage with the rack gear teeth 18b of the endless belt 17.

To the output axis 55a of the motor 55, a large spur gear 59 is connected. A shown in FIG. 3, each of the gear assemblies 50 is further provided with a small spur gear 54 engaged with the larger spur gear 59 and a bevel gear 53 connected to a shaft 54a of the small spur gear 54 and engaged with the bevel gear 51a of the driving roller 51.

Consequently, when the motor shaft 55a revolves by driving the motor 55, the bevel gear 51a will revolve, via the large spur gear 59, the spur gear 54, and the bevel gear 53, and therefore the pulley 51b will also revolve and the endless belts 17 will travel.

The driving unit casing 70 has a cylindrical shape larger in diameter than the insertion tube 9, as shown in FIG. 1. As shown in FIGS. 1 and 3, the casing 70 is provided with a motor storage section 71 in which the motor 55 is stored; a gear assembly storage section 72 in which the gear assemblies 50 are stored; and a guide pipe hole section 73 at which the guide pipe holes 73a and 73b are formed, which are arranged in order away from the insertion tube 9.

As shown in FIG. 3, the motor storage section 71 is liquid-tightly separated from the gear assembly storage section 72. The output axis 55a of the motor 55 protrudes into the gear assembly storage section 72 via a seal.

The gear assembly storage section 72 is openable and closeable by a lid 75 as shown in FIG. 1. When the lid 75 is opened, the gears 51, 53 and 54 constituting the gear assembly 50 are exposed so as to be cleaned easily. And, it makes possible to attach or detach the gears, facilitating exchange of the gear. And, it makes also possible to clean the driving unit easily so that it become unnecessary to remove the endless belt after the colonoscopic examination.

When the motor 55 is driven to rotate the pulley 51b counterclockwise in the figure, the exterior portion 17a of the endless belt 17 engaged with the pulley 51b will travel to the left direction in the figure. When the exterior portion 17a of the endless belt 17 is in contact with the inner wall of the colon, the insertion tube 9 will be propelled forward to the right direction in FIG. 8 by friction between the endless belts 17 and the inner wall of the colon. When the insertion tube 9 is propelled in the direction opposite to the insertion direction, the motor 55 is driven to rotate clockwise.

What is claimed is:

1. A self-propelled colonoscope comprising:
a tubular flexible insertion portion to be inserted into a colon;
an endless belt arranged around a circulating path on the inner wall and a part of the outer wall of the flexible insertion portion;
a driving mechanism to drive the endless belt;
guide hooks arranged around the circulating path on the outer wall of the flexible insertion portion; and
a guide pipe extending along the circulating path on the inner wall of the flexible insertion portion and guiding the endless belt,
wherein the circulating path on the outer wall of the flexible section at the distal end portion of the flexible insertion portion is 10 to 60 cm in length.

2. The self-propelled colonoscope according to claim 1,
wherein the endless belt travels through the guide pipes, arranged on the inner wall of the flexible insertion portion, other than the circulating path on the outer wall of the flexible insertion portion at the distal end portion of the flexible insertion portion.

3. A self-propelled colonoscope comprising:
a tubular flexible insertion portion to be inserted into a colon;
an endless belt arranged around a circulating path on the inner wall and a part of the outer wall of the flexible insertion portion;
a driving mechanism to drive the endless belt; and
a guide pipe extending along the circulating path on the inner wall of the flexible insertion portion and guiding the endless belt,
wherein the circulating path on the outer wall of the flexible insertion portion extends only along the distal end portion of the flexible insertion portion, and
the endless belt travels through the guide pipes forward and backward, arranged on the inner wall of the flexible insertion portion, other than the circulating path on the outer wall of the flexible insertion portion at the distal end portion of the flexible insertion portion.
